(19) United States
(12) Patent Application Publication
(10) Pub. No.: US 2014/0135830 A1
(43) Pub. Date: May 15, 2014

(54) DUAL DELIVERY SYSTEMS, DEVICES, AND RELATED METHODS FOR BIOADHESIVES

(71) Applicant: ST. JUDE MEDICAL PUERTO RICO LLC, Caguas, PR (US)

(72) Inventors: Edward E. Parsonage, St. Paul, MN (US); Martha Escobar, Jordan, MN (US); Zachary J. Tegels, Minneapolis, MN (US); Russell D. Terwey, St. Michael, MN (US); Troy T. White, Maple Grove, MN (US); Bernhard Kaeferlein, Champlin, MN (US); Timothy M. McGlinch, St Paul, MN (US)

(73) Assignee: St. Jude Medical Puerto Rico LLC, Caguas, PR (US)

(21) Appl. No.: 13/778,701

(22) Filed: Feb. 27, 2013

Related U.S. Application Data
(60) Provisional application No. 61/726,347, filed on Nov. 14, 2012.

(51) Int. Cl. A61B 17/00

(52) U.S. Cl. CPC A61B 17/0057
USPC 606/214

(57) ABSTRACT

A manifold for the delivery of bioadhesive sealant may comprise an inlet, a first outlet, a second outlet, and at least one valve positioned and configured to facilitate selective fluid communication between the inlet and the first outlet and the inlet and the second outlet. A syringe for delivering a bioadhesive sealant may comprise at least one barrel, at least one plunger, and at least one feature positioned and configured to prevent movement of the at least one plunger relative to the at least one barrel after a first predetermined quantity of bioadhesive sealant has been delivered. A bioadhesive sealant delivery system may comprise a manifold, a syringe, and a catheter, wherein a first lumen of the catheter is coupled to a first outlet of the manifold and a second lumen of the catheter coupled to a second outlet of the manifold.

### RELATED APPLICATION

This claims the benefit of U.S. Provisional Application No. 61/726,347, filed 14 Nov. 2012, which is hereby incorporated by reference herein in its entirety.

### TECHNICAL FIELD

The present disclosure relates generally to methods and systems for delivering bioadhesive sealants, and more particularly, to methods and systems for delivering bioadhesive sealants in multiple quantities over a period of time.

### BACKGROUND

Various surgical procedures are routinely carried out intravascularly or intraluminally. For example, in the treatment of vascular disease, such as arteriosclerosis, it is a common practice to access the artery and insert an instrument (e.g., a balloon or other type of catheter) to carry out a procedure within the artery. Such procedures usually involve the percutaneous puncture of the artery so that an insertion sheath may be placed in the artery and thereafter instruments (e.g., catheters) may pass through the sheath to an operative position within the artery. Intravascular and intraluminal procedures unavoidably present the problem of stopping the bleeding at the percutaneous puncture after the procedure has been completed and after the instruments (and any insertion sheaths used therewith) have been removed. Bleeding from puncture sites, particularly in the case of femoral arterial punctures, is typically stopped by utilizing vascular closure devices.

While there are a variety of prior art devices and techniques for closing such punctures, one primary problem is insuring a complete seal of the puncture. One technique includes the use of a bioadhesive sealant material to seal the puncture. Some types of bioadhesive sealant materials must be activated prior to use, and should be activated just prior to use in order to avoid premature activation of the bioadhesive sealant material. The handling and activation of bioadhesive sealant materials for use in vascular and other tissue puncture closure applications present a number of challenges, particularly when using bioadhesive sealant components that have a relatively short set time. For example, after a quantity of bioadhesive sealant is delivered through a lumen of a catheter, the bioadhesive sealant may become set within the lumen and prevent additional bioadhesive sealant from being delivered therethrough.

In view of the foregoing, improved systems, devices, and methods for delivering bioadhesive sealants would be desirable. Additionally, systems, devices, and methods for delivering bioadhesive sealants having a relatively short set time, and for delivering bioadhesive sealants in multiple doses, would be desirable.

### SUMMARY

One aspect of the present disclosure relates to manifolds for the delivery of bioadhesive sealant, which comprise an inlet, a first outlet, a second outlet, and at least one valve positioned and configured to facilitate selective fluid communication between the inlet and the first outlet and the inlet and the second outlet.

The inlet may comprise a first flow path and a second flow path, the first flow path being separate from the second flow path. The first outlet may comprise a first flow path and a second flow path, and the second outlet comprises a first flow path and a second flow path, the first flow path of each of the first and second outlets being positioned for selective fluid communication with the first flow path of the inlet and the second flow path of each of the first and second outlets being positioned for selective fluid communication with the second flow path of the inlet. The first outlet may comprise a mixing chamber in fluid communication with the first and second flow paths of the first outlet, and the second outlet may comprise a mixing chamber in fluid communication with the first and second flow paths of the second outlet.

The inlet may comprise at least one opening sized and configured for coupling to a syringe. The first outlet may comprise an opening sized and configured for coupling to a first lumen of a catheter and the second outlet may comprise an opening sized and configured for coupling to a separate second lumen of the catheter. The valve may comprise a first portion of the manifold, which comprises the inlet, slidable relative to a second portion of the manifold. The second portion of the manifold comprises the first and second outlets. The valve may comprise a first portion of the manifold, which comprises the inlet, rotatable relative to a second portion of the manifold. The second portion of the manifold comprises the first and second outlets.

The valve may comprise a three-way valve positioned between the inlet and the first and second outlets. The three-way valve may comprise a first flow path and a second flow path, the first flow path being configured to selectively provide fluid communication between the first flow path of the inlet and the first flow path of the first outlet, and the first flow path of the inlet and the first flow path of the second outlet. The second flow path may be configured to selectively provide fluid communication between the second flow path of the inlet and the second flow path of the first outlet, and the second flow path of the inlet and the second flow path of the second outlet.

Another aspect of the present disclosure relates to syringes for delivering a bioadhesive sealant, which comprises at least one barrel, at least one plunger, and at least one feature positioned and configured to prevent movement of the at least one plunger relative to the at least one barrel after a first predetermined quantity of bioadhesive sealant has been delivered.

The at least one feature may comprise at least one tab, which provides mechanical interference between the at least one plunger and the at least one barrel when the first predetermined quantity of bioadhesive sealant has been delivered. The at least one tab may be configured to allow the at least one plunger to move relative to the at least one barrel to deliver a second predetermined quantity of bioadhesive sealant upon a force being applied to the at least one tab.

An additional aspect of the present disclosure relates to bioadhesive sealant delivery systems, which comprises a manifold, a syringe, and a catheter. The manifold comprises an inlet, a first outlet, a second outlet, and at least one valve positioned and configured to facilitate selective fluid communication between the inlet and the first outlet and the inlet and the second outlet. The syringe is coupled to the inlet of the manifold, a first lumen of the catheter is coupled to the first outlet of the manifold, and a second lumen of the catheter is coupled to the second outlet of the manifold.

The catheter is configured to deliver a first volume of bioadhesive sealant through the first lumen to a vessel puncture, and deliver a second volume of bioadhesive sealant through the second lumen to the vessel puncture. The catheter comprises an expandable member configured to temporarily seal the vessel puncture. The inlet comprises a first flow path and a second flow path, the first flow path being separate from the second flow path. The first outlet comprises a first flow path and a second flow path and the second outlet comprises a first flow path and a second flow path, the first flow path of each of the first and second outlets positioned for selective fluid communication with the first flow path of the inlet and the second flow path of each of the first and second outlets positioned for selective fluid communication with the second flow path of the inlet. The first outlet comprises a mixing chamber in fluid communication with the first and second flow paths of the first outlet, and the second outlet comprises a mixing chamber in fluid communication with the first and second flow paths of the second outlet.

A further aspect of the present disclosure relates to methods of delivering bioadhesive sealants. Such methods comprise delivering a first quantity of bioadhesive sealant from an inlet of a manifold to a first outlet of the manifold, operating a valve of the manifold, and delivering a second quantity of bioadhesive sealant from the inlet of the manifold to a second outlet of the manifold.

The methods may further comprise delivering the first quantity of bioadhesive sealant from the first outlet of the manifold to a first lumen of a catheter, and delivering the second quantity of bioadhesive sealant from the second outlet of the manifold to a second lumen of the catheter. The methods may further comprise delivering the first quantity of bioadhesive sealant from a syringe to the inlet of the manifold, and delivering the second quantity of bioadhesive sealant from the syringe to the inlet of the manifold. The methods may further comprise depressing at least one tab of the syringe after delivering the first quantity of bioadhesive sealant and before delivering the second quantity of bioadhesive sealant. Operating the valve of the manifold may comprise sliding a first portion of the manifold relative to a second portion of the manifold. Operating the valve of the manifold may comprise rotating a first portion of the manifold relative to a second portion of the manifold.

The foregoing and other features, utilities, and advantages of the invention will be apparent from the following detailed description of the invention with reference to the accompanying drawings.

### BRIEF DESCRIPTION OF THE DRAWINGS

The accompanying drawings illustrate various embodiments of the present disclosure and are a part of the specification. The illustrated embodiments are merely examples of the present disclosure and do not limit the scope of the invention.

FIG. 1 is a perspective view of a manifold for delivering bioadhesives having a three-way valve positioned in a first position, according to an embodiment of the present disclosure.

FIG. 2 is a perspective view of the manifold of FIG. 1 having the three-way valve positioned in a second position

FIG. 3 is a perspective view of a manifold for delivering bioadhesives having a three-way valve with bifurcated flow paths positioned in a first position, according to an embodiment of the present disclosure.

FIG. 4 is a perspective view of the manifold of FIG. 3 having the three-way valve with bifurcated flow paths positioned in a second position.

FIG. 5 is a front view of a double-barrel syringe including tabs to facilitate delivering two known quantities of bioadhesive sealant, according to an embodiment of the present disclosure.

FIG. 6 is a front view of the double-barrel syringe of FIG. 5, after a first predetermined amount of bioadhesive sealant has been delivered.

FIG. 7 is a front view of the double-barrel syringe of FIG. 5, after the tabs have been depressed and a second predetermined amount of bioadhesive sealant has been delivered.

FIGS. 8-11 illustrate the use of a syringe and manifold, such as shown in FIGS. 3-7, with a vascular closure device and a sheath to seal a vessel puncture, according to an embodiment of the present disclosure.

FIG. 12 is a cross-sectional view of a manifold having a first portion rotatable relative to a second portion, according to an embodiment of the present disclosure.

FIG. 13 is a cross-sectional view of the manifold of FIG. 12, wherein the first portion has been rotated to a second position.

FIG. 14 is a cross-sectional view of a manifold having a first portion slidable relative to a second portion, according to an embodiment of the present disclosure.

FIG. 15 is a cross-sectional view of the manifold of FIG. 14, wherein the first portion has been slid to a second position.

### Throughout the drawings, identical reference numbers designate similar, but not necessarily identical, elements.

### DETAILED DESCRIPTION

The systems disclosed herein may be used to close or seal percutaneous punctures made through the body tissue of a patient to gain access to a body cavity of a patient. Access through these percutaneous punctures allows a physician to carry out various procedures in or through the body cavity for examination, surgery, treatment and the like. While not meant to be limiting, the systems are illustrated being used to seal percutaneous punctures that provide access to blood vessels in patients for various procedures. It will be appreciated that the systems are applicable to other procedures requiring sealing of a puncture through body tissue into a cavity including, for example, laparoscopic surgery and other microscopic surgery techniques using a relatively small incision.

As used in this specification and the appended claims, the terms “engage” and “engagable” are used broadly to mean interlock, mesh, or contact between two structures or devices. Likewise “disengage” or “disengagable” means to remove or capable of being removed from interlock, mesh, or contact. The words “including” and “having,” as well as their derivatives, as used in the specification, including the claims, have the same meaning as the word “comprising.”

The general structure and function of tissue closure devices used for sealing a tissue puncture in an internal tissue wall accessible through an incision in the skin are well known in the art. Applications of closure devices including those implementing principles described herein include closure of a percutaneous puncture or incision in tissue separating two internal portions of a living body, such as punctures or incisions in blood vessels, ducts or lumens, gall bladders, livers, hearts, etc.

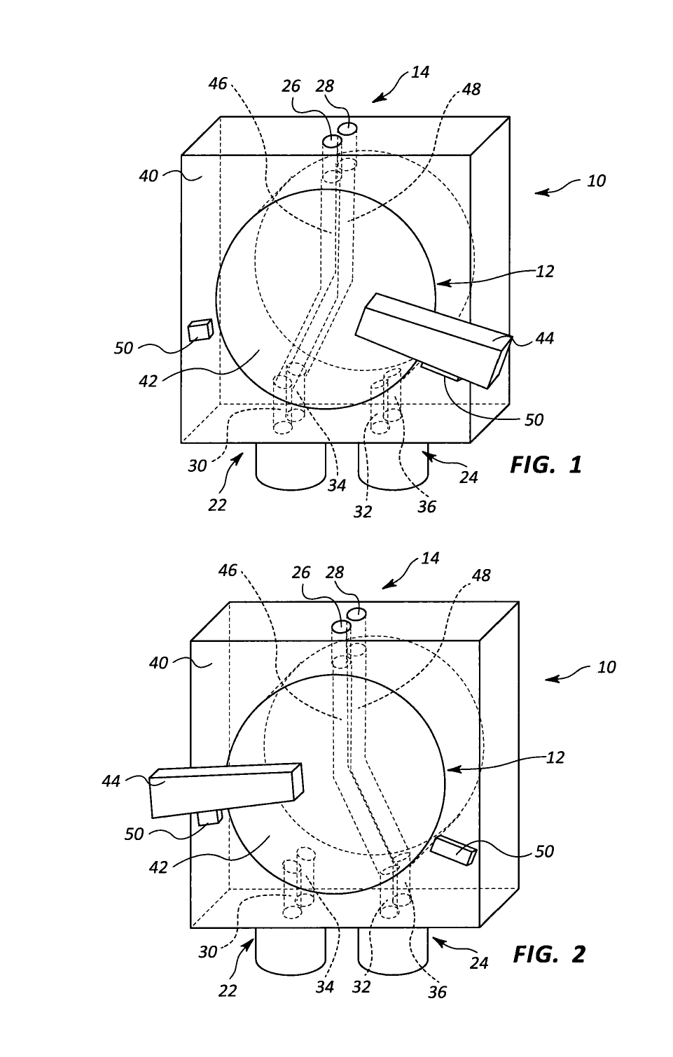

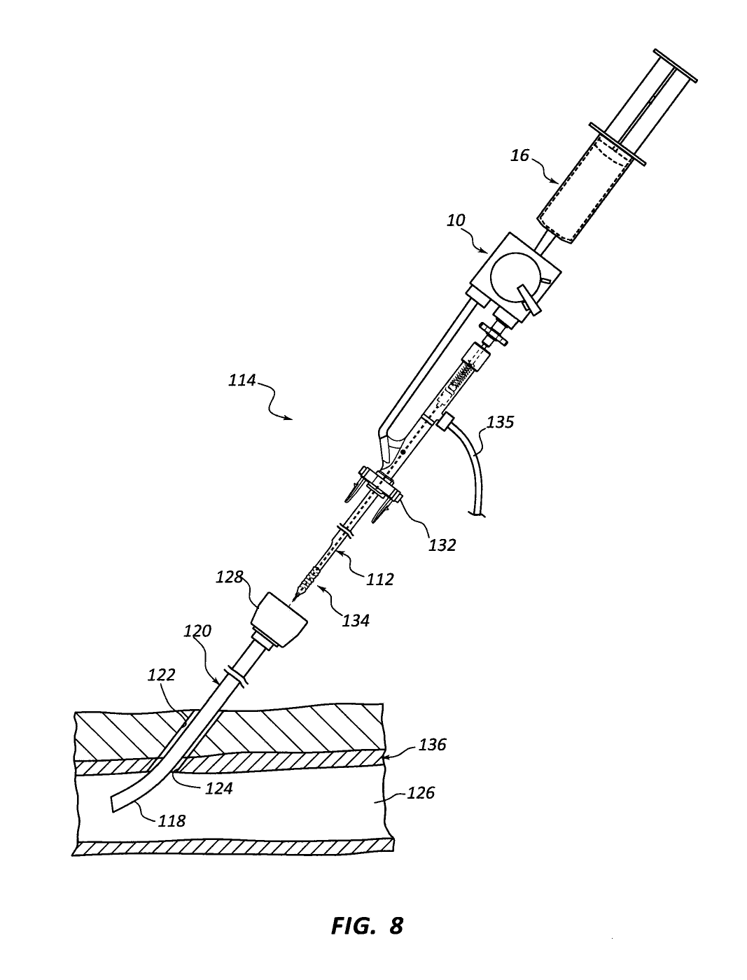

In some embodiments, a bioadhesive sealant delivery system may include a manifold 10 including one or more valves 12, such as shown in FIGS. 1-2. The manifold 10 may have an inlet 14 configured to receive a bioadhesive sealant source, such as a double barrel syringe 16 comprising a bioadhesive sealant precursor 18 and an activator 20 as shown in FIGS. 5-7. The manifold 10 may additionally include a first outlet 22 and a separate second outlet 24. The first outlet 22 may be configured to couple with a catheter, such as shown in FIGS. 8-11, and provide a bioadhesive sealant, such as a bioadhesive sealant comprising a mixture of a bioadhesive sealant precursor 18 and an activator 20, to a first lumen of the catheter. Similarly, the second outlet 24 may also be configured to couple with the catheter and provide a bioadhesive sealant to a separate second lumen of the catheter.

The inlet 14 of the manifold 10 may comprise a first opening to a first fluid flow path 26 and a second opening to a separate second fluid flow path 28. Similarly, each of the first and second outlets 22, 24 of the manifold 10 may include a first opening to a first fluid flow path 30, 32 and a second opening to a separate second fluid path 34, 36. Additionally, the manifold 10 may include at least one valve 12 configured for selectively providing fluid communication between the first fluid flow path 26 of the inlet 14 and one of the first fluid flow path 30 of the first outlet 22 and the first fluid flow path 32 of the second outlet 24. Similarly, the one or more valves 12 of the manifold 10 may be configured to substantially simultaneously selectively provide fluid communication between the second fluid flow path 28 of the inlet 14 and one of the second fluid flow path 34 of the first outlet 22 and the second fluid flow path 36 of the second outlet 24.

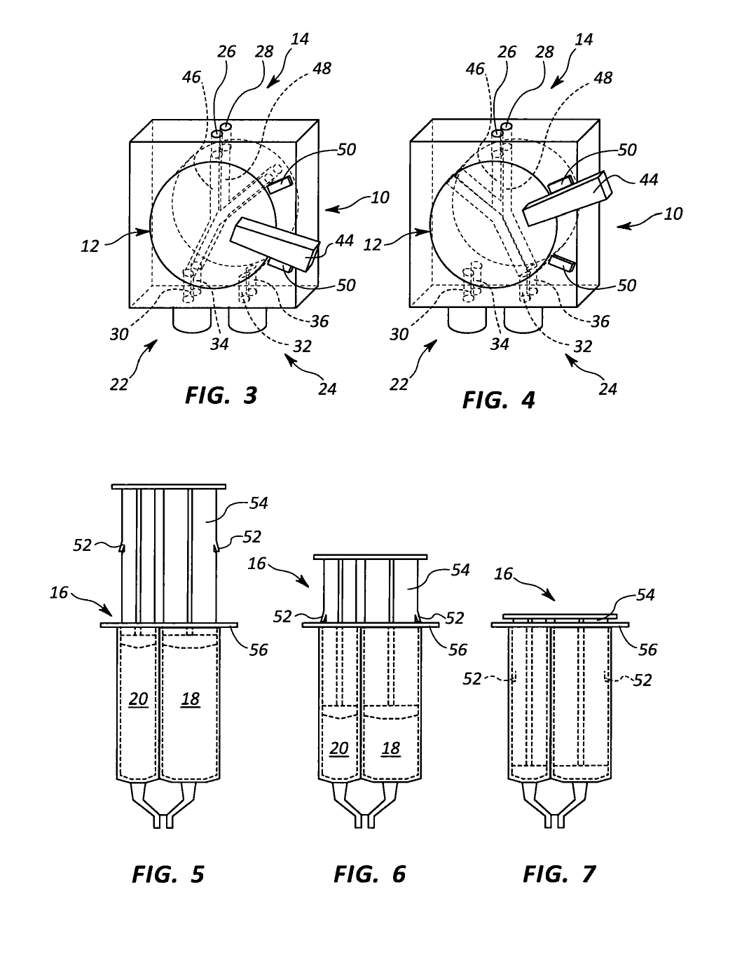

In some embodiments, the manifold 10 may comprise a housing 40 and a three-way valve 12. The housing 40 may include the respective fluid flow paths 26, 28, 30, 32, 34, 36 of the inlet 14, the first outlet 22 and the second outlet 24. The three-way valve 12 may be positioned at least partially within the housing 40 and rotatable relative to the housing 40. The three-way valve 12 may include a body 42, a handle 44 coupled to the body 42, a first fluid flow path 46 extending through the body 42, and a separate second fluid flow path 48 extending through the body 42. The housing 40 may include a plurality of visual indicators or stops 50, which may facilitate the positioning of the three-way valve 12 in a first position, as shown in FIGS. 1 and 3, and a second position, as shown in FIGS. 2 and 4. For example, the housing 40 may include stops 50 that comprise protrusions from a surface of the housing 40 that may be sized and configured to prevent the handle 44 of the three-way valve 12 to rotate past the stops 50 due to mechanical interference between each stop 50 and the handle 44.

As shown in FIGS. 1 and 3, when the three-way valve 12 is positioned in the first position, the first and second fluid flow paths 46, 48 of the three-way valve 12 are aligned with the respective first and second fluid flow paths 26, 28 of the inlet 14. Additionally, when the three-way valve is positioned in the first position, the first and second fluid flow paths 46, 48 of the three-way valve 12 are aligned with the respective first and second fluid flow paths 30, 34 of the first outlet 22. The first and second fluid flow paths 46, 48 of the three-way valve 12 are not aligned with, and are not in fluid communication with, the respective first and second fluid flow paths 32, 36 of the second outlet 24 when the three-way valve 12 is positioned in the first position.

As shown in FIGS. 2 and 4, when the three-way valve 12 is positioned in the second position the first and second fluid flow paths 46, 48 of the three-way valve 12 are aligned with the respective first and second fluid flow paths 26, 28 of the inlet 14. Additionally, when the three-way valve 12 is positioned in the second position, the first and second fluid flow paths 46, 48 of the three-way valve 12 are aligned with the respective first and second fluid flow paths 32, 36 of the second outlet 24. The first and second fluid flow paths 46, 48 of the three-way valve 12 are not aligned with, and are not in fluid communication with, the respective first and second fluid flow paths 30, 34 of the first outlet 22 when the three-way valve 12 is positioned in the second position. Accordingly, the three-way valve 12 may be utilized to selectively provide fluid communication between the inlet 14 and one of the first outlet 22 and the second outlet 24 by movement, such as with the handle 44, between the first position and the second position.

In some embodiments, the first and second fluid flow paths 46, 48 within the valve body 42 may each be configured as a single continuous flow path, such as shown in FIGS. 1 and 2. In further embodiments, such as shown in FIGS. 3 and 4, a three-way valve 12 may include first and second fluid flow paths 46, 48 that are bifurcated. For example, each of the first and second fluid flow paths 46, 48 through the body 42 of the three-way valve 12 may be generally Y-shaped. Bifurcated first and second fluid flow paths 46, 48 may allow a relatively small rotational movement of the valve 12 between the first position and the second position, whereas single continuous first and second fluid flow paths 46, 48 may provide relative simplicity in manufacture.

The syringe 16, shown in FIGS. 5-7, may be a double barrel syringe 16 having a bioadhesive sealant precursor 18 located within a first barrel and an activator 20 located within a second barrel. The syringe 16 may include a feature that facilitates the delivery of two known quantities of bioadhesive sealant. For example, the syringe 16 may include a tab 52 on one or more plungers 54 positioned to stop the depression of the plungers 54 after a predetermined amount of bioadhesive sealant (i.e., a certain amount of bioadhesive sealant precursor 18 and activator 20) has been delivered, as shown in FIG. 6. For example, tabs 52 may mechanically interfere with a body 56 of the syringe 16 after the plungers 54 have traveled a predetermined distance relative to the body 56 of the syringe 16. The one or more tabs 52 may then be depressed by an operator to allow further depression of the plungers 54 to deliver the remaining bioadhesive sealant, as shown in FIG. 7. In further embodiments, other features may be positioned on a plunger 54 that may cooperate with features on the body 56 of the syringe 16 to facilitate the delivery of multiple predetermined amounts of bioadhesive sealant from the syringe 16.

In operation, a prepared syringe 16 may be coupled to a manifold 10, and the manifold 10 (e.g., see embodiment of FIGS. 3-4) may be coupled to a proximal end of a delivery tube 112 of a vascular closure device 114, as shown in FIG. 8. The first outlet 22 of the manifold 10 is connected to a first lumen 104 of the delivery tube 112. The second outlet 24 of the manifold 10 is connected to a second lumen 144 of the delivery tube 112.

As further shown in FIG. 8, a distal end 118 of a sheath 120 may be advanced through a tissue tract 122 and a vessel puncture 124 and into a vessel lumen 126. The vascular closure device 114 may be aligned with an opening into a hub 128 of the sheath 120 for insertion into the sheath.

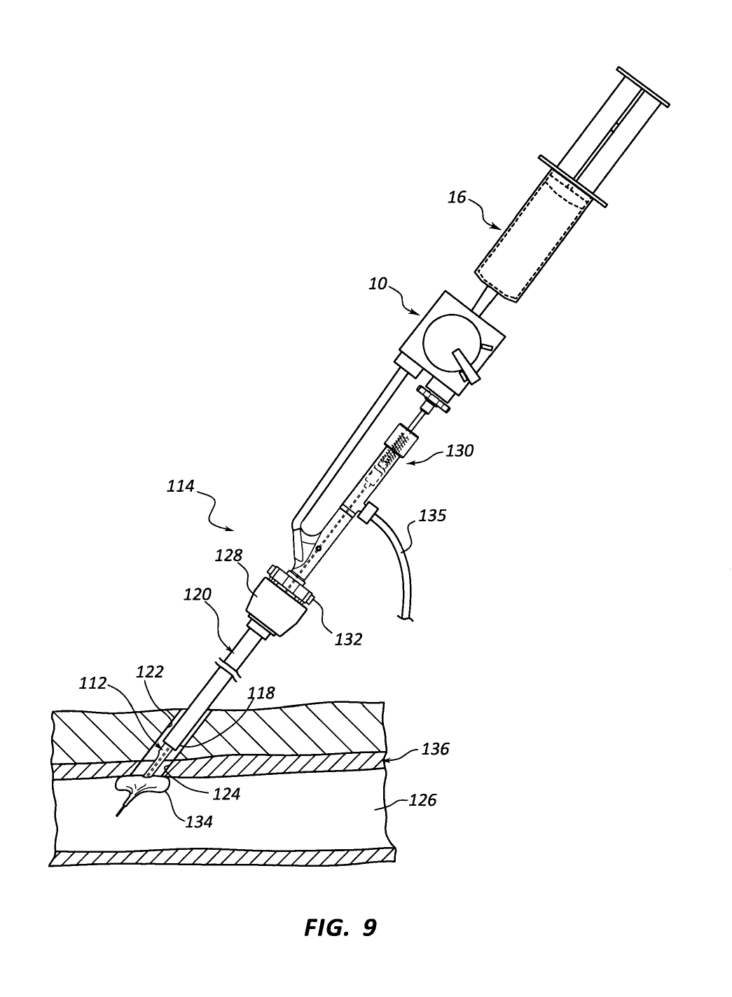

Referring to FIG. 9, the delivery tube 112 ma y be advanced through the sheath 120 and a latch 132 may be connected to the hub 128 of the sheath 120. A balloon 134 may be inflated by delivering a volume of inflation fluid from an inflation fluid source 135, through a housing of a balloon location device 130, through an inflation fluid lumen of the delivery tube 112, and into the balloon 134. The vascular closure device 114 and sheath 120 may then be retracted (e.g., withdrawn proximally) to bring the inflated balloon 134 into contact with an inner surface of the vessel 136 adjacent to the vessel puncture 124. Accordingly, the inflated balloon 134 may provide a temporary seal with the vessel 136 to limit blood flow through the vessel puncture 124 from within the vessel lumen 126.

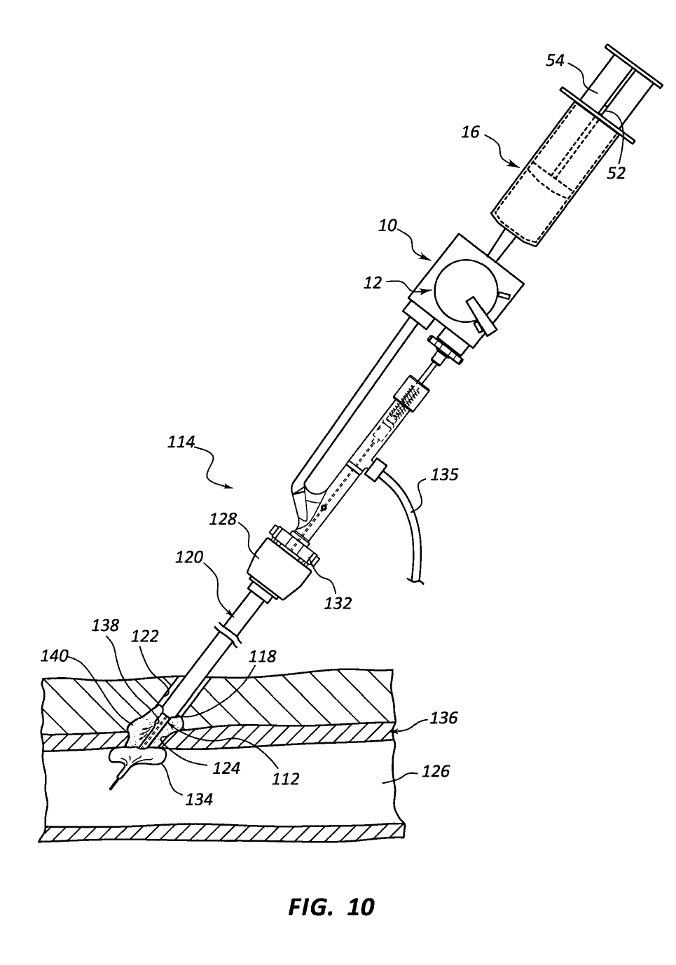

Referring to FIG. 10, plungers 54 of the syringe 16 may then be advanced, with the valve 12 of the manifold 10 located in the first position. As the plungers 54 of the syringe 16 are advanced, a bioadhesive sealant precursor 18 from the first barrel of the syringe 16 (see FIG. 5) may be directed through the first fluid flow path 26 of the inlet 14 (see FIG. 3). Substantially simultaneously, the activator 20 may be directed from the second barrel of the syringe 16 (see FIG. 5) through the second fluid flow path 28 of the inlet 14 (see FIG. 3). The valve 12, positioned in the first position, may then direct the bioadhesive sealant precursor 18 and the activator 20 to the first outlet 22 via separate flow paths (see FIG. 3).

Within a mixing chamber of the first outlet 22, or optionally, upon exiting the first outlet 22, the bioadhesive sealant precursor 18 and the activator 20 may mix together to form an uncured bioadhesive sealant.

Referring again to FIG. 10, the uncured bioadhesive sealant is then delivered through the first lumen 104 of the delivery tube 112, and out a distal opening 138, to the vessel puncture 124 and tissue tract 122. The bioadhesive sealant may form a bioadhesive plug 140 that may seal closed the vessel puncture 124 and tissue tract 122 from outside of the vessel 136. The bioadhesive sealant forming the bioadhesive plug 140 may be allowed to at least partially cure into a solid or semi-solid state that limits movement of the bioadhesive sealant of the bioadhesive plug 140 into the vessel lumen 126 upon deflating the balloon 134. Additionally, the bioadhesive sealant remaining within the first lumen of the catheter may also cure, preventing further uncured bioadhesive sealant from being delivered through the first lumen.

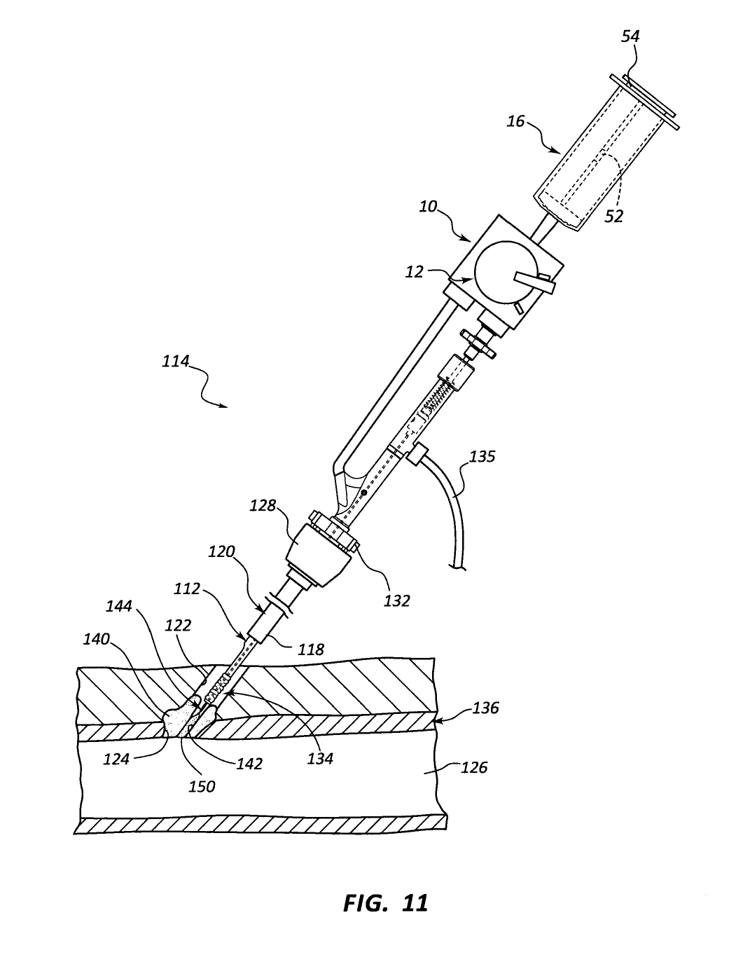

Referring to FIG. 11, the balloon 134 may then be deflated by withdrawing the inflation fluid through the inflation fluid lumen of the delivery tube 112. The vascular closure device 114 and sheath 120 may then be further retracted or withdrawn, so that the delivery tube 112 may be positioned proximal to the bioadhesive plug 140. A tract 142 may be defined within the bioadhesive plug 140 after removal of the delivery tube 112. The tract 142 may be filled by delivering a second bioadhesive sealant via the second lumen 144.

To deliver the second quantity of bioadhesive sealant to the tract 142, the valve 12 of the manifold 10 may be rotated to the second position. After the first quantity of bioadhesive sealant is delivered, the tabs 52 on the syringe 16 may prevent the further depression of the plungers 54. Accordingly, the tabs 52 may be depressed on the syringe 16 to allow the plungers 54 of the syringe 16 to be further depressed to deliver further bioadhesive sealant precursor 18 from the first barrel of the syringe 16 (see FIG. 6) through the first fluid flow path 26 of the inlet 14 (see FIG. 4). Substantially simultaneously, further activator 20 may be directed from the second barrel of the syringe 16 (see FIG. 6) through the second fluid flow path 28 of the inlet 14 (see FIG. 4). The valve 12, located in the second position, may then direct the bioadhesive sealant precursor 18 and the activator 20 to the second outlet 24 via separate first and second fluid flow paths 46, 48 (see FIG. 4).

Within the mixing chamber of the second outlet 24, or optionally, upon exiting the second outlet 24 (see FIG. 4), the bioadhesive sealant precursor 18 and the activator 20 (see FIG. 6) may mix together to form uncured bioadhesive sealant. As shown in FIG. 11, the uncured bioadhesive sealant may then be directed through the second lumen 144 to the tract 142. The second bioadhesive sealant may then form into a second bioadhesive plug 150 within the tract 142 to provide further sealing of the vessel puncture 124.

After delivering the second bioadhesive sealant and forming the second bioadhesive plug 150, the entire vascular closure device 114 and sheath 120 may be removed from the tissue tract 122 and the sealing procedure may be complete.

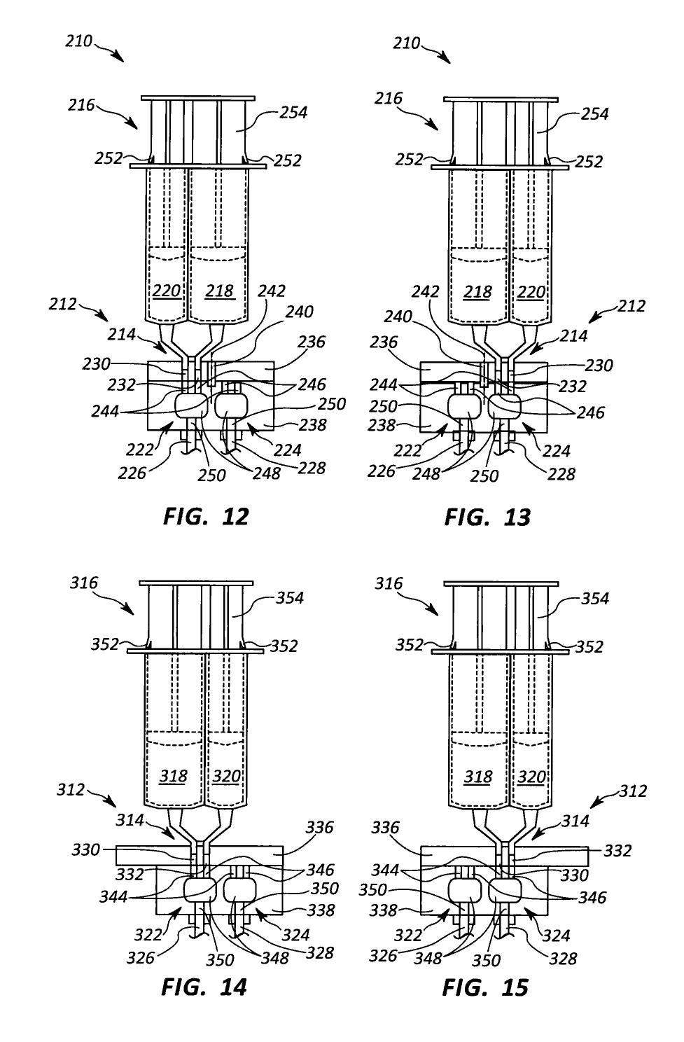

In additional embodiments, a bioadhesive sealant delivery system 210, such as for sealing a tissue puncture, may include a manifold 212 (e.g., see FIGS. 12 and 13), and may have an inlet 214 configured to receive a bioadhesive sealant source, such as a double barrel syringe 216 comprising a bioadhesive sealant precursor 218 in a first barrel and an activator 220 in a second barrel. The manifold 212 may additionally include a first outlet 222 and a separate second outlet 224. The first outlet 222 may be configured to couple with a first lumen 226 of a catheter, and provide a bioadhesive sealant, such as a bioadhesive sealant comprising a mixture of the bioadhesive sealant precursor 218 and the activator 220, to the first lumen 226 of the catheter. Similarly, the second outlet 224 may be configured to couple with a separate second lumen 228 of the catheter and provide a bioadhesive sealant to the second lumen 228 of the catheter.

The inlet 214 of the manifold 212 may comprise a first opening to a first fluid flow path 230 and a second opening to a separate second fluid flow path 232. Additionally, the manifold 212 may include at least one valve configured to selectively provide fluid communication between the inlet 214 and the first and second outlets 222 and 224.

In one embodiment, as shown in FIGS. 12 and 13, the inlet 214 may be positioned in a first portion 236 of the manifold 212 that may rotate relative to a second portion 238 of the manifold 212, and the first and second outlets 222 and 224 may be positioned in the second portion 238 of the manifold 212. Accordingly, the first portion 236 of the manifold 212 may be positioned to a first position and the inlet 214 may be aligned with the first outlet 222, as shown in FIG. 12. Additionally, the first portion 236 of the manifold 212 may be rotatable from the first position to a second position to align the inlet 214 with the second outlet 224, as shown in FIG. 13.

The first portion 236 of the manifold 212 may include the first flow path 230 and the second flow path 232 of the inlet 214 extending therethrough. The first portion 236 of the manifold 212 may also include openings into the first and second flow paths 230 and 232 of the inlet 214 sized and configured to couple the syringe 216 to the inlet 214. A pivot joint 240, such as one or more of a screw, a pin, a shoulder bolt, and another structure, may join the first portion 236 of the manifold 212 to the second portion 238 of the manifold 212 and facilitate the rotation of the first portion 236 of the manifold 212 relative to the second portion 238 of the manifold 212 about a pivot axis 242.

Each of the first and second outlets 222 and 224 may be located in the second portion 238 of the manifold 212 and may include a first flow path 244, a second flow path 246, a mixing chamber 248 (e.g., a mixing tip), and a third flow path 250. Each of the first flow paths 244 of the first and second outlets 222 and 224 may be positioned and configured for selective alignment with the first flow path 230 of the inlet 214 and the second flow paths 246 of the first and second outlets 222 and 224 may be positioned and configured for selective alignment with the second flow path 232 of the inlet 214. Each mixing chamber 248 may be in fluid communication with the first, second and third flow paths 244, 246, and 250 of the first and second outlets 222 and 224, respectively, and positioned between the third flow path 250 and the first and second flow paths 244 and 246. Each of the third flow paths 250 of the first and second outlets 222 and 224 may extend to a respective opening of the second portion 238 of the manifold 212, which may each be sized and configured to couple to a respective first and second lumen 226, 228 of the catheter.

In operation, a first quantity of bioadhesive sealant may be delivered to the first lumen 226 of the catheter via the first outlet 222 of the manifold 212. To deliver the first quantity of bioadhesive sealant, the syringe 216 may be coupled to the inlet 214 of the manifold, and the first portion 236 of the manifold 212 may be located in the first position (see FIG. 12). The plungers 254 of the syringe 216 may then be depressed to deliver a bioadhesive sealant precursor 218 from the first barrel of the syringe 216 through the first flow path 230 of the inlet 214. Substantially simultaneously, an activator 220 may be directed from the second barrel of the syringe 216 through the second flow path 232 of the inlet 214. The first portion 236 of the manifold 212, positioned in the first position, may then direct the bioadhesive sealant precursor and the activator to the first outlet 222 via separate first and second flow paths 230, 232, respectively.

Upon exiting the first outlet 222, the bioadhesive sealant precursor and the activator may mix together to form an uncured bioadhesive sealant. The uncured bioadhesive sealant may then be directed through the first lumen 226 of the catheter and into a target site, such as a percutaneous puncture. After a period of time, the uncured bioadhesive sealant may cure, becoming firm. Accordingly, the bioadhesive sealant may provide a bioadhesive plug at the target site. Additionally, the bioadhesive sealant within the first lumen 226 of the catheter may also become firm over the period of time, preventing further uncured bioadhesive sealant from being delivered through the first lumen 226.

To deliver a second quantity of bioadhesive sealant to the target site, the first portion 236 of the manifold 212 may be rotated in the second position (see FIG. 13). After the first quantity of bioadhesive sealant is delivered, tabs 252 on the syringe 216 may prevent the further depression of the plungers 254. Accordingly, one or more tabs 252 may be depressed on the syringe 216 to allow the plungers 254 of the syringe 216 to be further depressed to deliver further bioadhesive sealant precursor 218 from the first barrel of the syringe 216 through the first flow path 230 of the inlet 214. Substantially simultaneously, further activator 220 may be directed from the second barrel of the syringe 216 through the second flow path 232 of the inlet 214. The first portion 236 of the manifold 212, located in the first position, may then direct the bioadhesive sealant precursor and the activator to the second outlet 224 via separate first and second flow paths 230, 232.

Upon exiting the second outlet 224, the bioadhesive sealant precursor 218 and the activator 220 may mix together to form an uncured bioadhesive sealant. The uncured bioadhesive sealant may then be directed through the second lumen 228 of the catheter and into the target site. After a period of time, the uncured bioadhesive sealant may cure, becoming firm. Accordingly, the second quantity of bioadhesive sealant may be provided at the target site, facilitating the use of a bioadhesive sealant having a relatively short curing time.

In an additional embodiment, as shown in FIGS. 14 and 15, a first inlet 314 may be positioned in a first portion 336 of a manifold 312 that may be linearly movable relative to a second portion 338 of the manifold 312, and first and second outlets 322, 324 may be positioned in the second portion 338 of the manifold 312. Accordingly, the first portion 336 of the manifold 312 may be positioned to a first position and the inlet 314 may be aligned with the first outlet 322, as shown in FIG. 14. Additionally, the first portion 336 of the manifold 312 may be linearly movable from the first position to a second position to align the inlet 314 with the second outlet 324, as shown in FIG. 15.

The first portion 336 of the manifold 312 may include a first flow path 330 and a second flow path 332 of the first inlet 314 extending therethrough. The first portion 336 of the manifold 312 may also include openings into the first and second flow paths 330, 332 of the inlet 314 sized and configured to couple one or more syringes 316 to the inlet 314, such as a double barrel syringe 316 comprising a bioadhesive sealant precursor 318 in a first barrel and an activator 320 in a second barrel. A linear slide, such as a dovetail slide, may join the first portion 336 of the manifold 312 to the second portion 338 of the manifold 312 and allow the first portion 336 to move linearly relative to the second portion 338.

Each of the first and second outlets 322, 324 located in the second portion 338 of the manifold 312 may include a first flow path 344, a second flow path 346, a mixing chamber 348 (e.g., a mixing tip), and a third flow path 350. Each of the first flow paths 344 of the first and second outlets 322, 324 may be positioned and configured for selective alignment with the first flow path 330 of the inlet 314, and the second flow paths 346 of the first and second outlets 322, 324 may be positioned and configured for selective alignment with the second flow path 332 of the inlet 314. Each mixing chamber 348 may be in fluid communication with the first, second and third flow paths 344, 346, 350 of the first and second outlets 322, 324, respectively, and positioned between the third flow path 350 and the first and second flow paths 344, 346. Each of the third flow paths 350 of the first and second outlets 322, 324 may extend to a respective opening of the second portion 338 of the manifold 312, which may each be sized and configured to couple a respective first or second lumen 326, 328 of a catheter thereto.

In operation, a first quantity of bioadhesive sealant may be delivered to a first lumen 326 of the catheter via the first outlet 322 of the manifold. To deliver the first quantity of bioadhesive sealant, the syringe 316 may be coupled to the inlet 314 of the manifold 312, and the first portion 336 of the manifold 312 may be located in the first position (see FIG. 14). The plungers 354 of the syringe 316 may then be depressed to deliver a bioadhesive sealant precursor 318 from the first barrel of the syringe 316 through the first flow path 330 of the inlet 314. Substantially simultaneously, an activator 320 may be directed from the second barrel of the syringe 316 through the second flow path 332 of the inlet 314. The first portion 336 of the manifold 312, positioned in the first position, may then direct the bioadhesive sealant precursor 318 and the activator 320 to the first outlet 322 via separate first and second flow paths 330, 332.

Within the mixing chamber 348 of the first outlet 322, or optionally, upon exiting the first outlet 322, the bioadhesive sealant precursor 318 and the activator 320 may mix together to form an uncured bioadhesive sealant. The uncured bioadhesive sealant may then be directed through the first lumen 326 of the catheter and into a target site, such as a percutaneous puncture. After a period of time, the uncured bioadhesive sealant may cure, becoming firm. Accordingly, the bioadhesive sealant may provide a bioadhesive plug at the target site. Additionally, the bioadhesive sealant within the first lumen 326 of the catheter may also become cured, preventing further uncured bioadhesive sealant from being delivered through the first lumen 326.

To deliver a second quantity of bioadhesive sealant to the target site, the first portion 336 of the manifold 312 may be slid relative to the second portion 338 of the manifold 312, to the second position (see FIG. 15). After the first quantity of bioadhesive sealant is delivered, tabs 352 on the syringe 316 may prevent the further depression of the plungers 354. Accordingly, one or more tabs 352 may be depressed on the syringe 316 to allow the plungers 354 to be further depressed to deliver further bioadhesive sealant precursor 318 from the first barrel of the syringe 316 through the first flow path 330 of the inlet 314 to the second outlet 324. Substantially simultaneously, further activator 320 may be directed from the second barrel of the syringe 316 through the second flow path 332 of the inlet 314 to the second outlet 324. The first portion 336 of the manifold 312, located in the second position, may direct the bioadhesive sealant precursor 318 and the activator 320 to the second outlet 324 via separate first and second flow paths 330, 332.

Within the mixing chamber of the second outlet, or optionally, upon exiting the second outlet, the bioadhesive sealant precursor and the activator may mix together to form an uncured bioadhesive sealant. The uncured bioadhesive sealant may then be directed through the second lumen 328 of the catheter and into the target site. After a period of time, the uncured bioadhesive sealant may cure, becoming firm. Accordingly, the second quantity of bioadhesive sealant may be provided at the target site, facilitating the use of a bioadhesive sealant having a relatively short curing time.

The sealants discussed herein may comprise a single component, or may comprise multiple sealant components that are mixed together. The multiple sealant components may further react together to form a cross-linked network. The sealant components may be naturally derived or synthetic. Some example synthetic components include polyethers such as polyethylene glycol, polypropylene glycol and polytetrahydrofuran. Other examples of synthetic components may include polyamine compositions such as polyvinylpyrrolidones, polyethylene imines and hydrogenated polyacrylonitriles. Other example sealant components include polyacrylic and methacrylic compounds such as polyacrylic acid. Example naturally derived components include protienaceous compositions such as albumin, collagen and polylysine. Other examples include carbohydrate compositions such polyhyaluronic acid. The sealant components may also contain reactive functional groups to promote chemical cross-linking. The sealant components may be cross-linked by any known method including, for example, condensation reactions, Michael addition, and free radical. Functional groups used for cross-linking may include, for example, thiols, acrylates, amines, succinimydyls and aldehydes, to name a few.

The preceding description has been presented only to illustrate and describe exemplary embodiments of the invention. It is not intended to be exhaustive or to limit the invention to any precise form disclosed. Many modifications and variations are possible in light of the above teaching. It is intended that the scope of the invention be defined by the following claims.

What is claimed is:

1. A manifold for delivery of bioadhesive sealant, comprising:
an inlet;
a first outlet;
a second outlet;
at least one valve positioned and configured to facilitate selective fluid communication between the inlet and the first outlet and the inlet and the second outlet.

2. The manifold of claim 1, wherein the inlet comprises a first flow path and a second flow path, the first flow path being separate from the second flow path.

3. The manifold of claim 2, wherein the first outlet comprises a first flow path and a second flow path and the second outlet comprises a first flow path and a second flow path, the first flow path of each of the first and second outlets positioned for selective fluid communication with the first flow path of the inlet and the second flow path of each of the first and second outlets positioned for selective fluid communication with the second flow path of the inlet.

4. The manifold of claim 3, wherein the first outlet comprises a mixing chamber in fluid communication with the first and second flow paths of the first outlet, and the second outlet comprises a mixing chamber in fluid communication with the first and second flow paths of the second outlet.

5. The manifold of claim 1, wherein the inlet comprises at least one opening sized and configured for coupling to a syringe.

6. The manifold of claim 1, wherein the first outlet comprises an opening sized and configured for coupling to a first lumen of a catheter and the second outlet comprises an opening sized and configured for coupling to a separate second lumen of the catheter.

7. The manifold of claim 1, wherein the at least one valve comprises a first portion of the manifold, which comprises the inlet, slidable relative to a second portion of the manifold, which comprises the first and second outlets.

8. The manifold of claim 1, wherein the at least one valve comprises a first portion of the manifold, which comprises the inlet, rotatable relative to a second portion of the manifold, which comprises the first and second outlets.

9. The manifold of claim 1, wherein the at least one valve comprises a three-way valve positioned between the inlet and the first and second outlets.

10. The manifold of claim 9, wherein the three-way valve comprises a first flow path and a second flow path, the first flow path configured to selectively provide fluid communication between the first flow path of the inlet and the first flow path of the first outlet, and the first flow path of the inlet and the first flow path of the second outlet; and the second flow path configured to selectively provide fluid communication between the second flow path of the inlet and the second flow path of the first outlet, and the second flow path of the inlet and the second flow path of the second outlet.

11. A syringe for delivering a bioadhesive sealant, comprising:
at least one barrel;
at least one plunger;
at least one feature positioned and configured to prevent movement of the at least one plunger relative to the at least one barrel after a first predetermined quantity of bioadhesive sealant has been delivered.

12. The syringe of claim 11, wherein the at least one feature comprises at least one tab which provides mechanical interference between the at least one plunger and the at least one barrel when the first predetermined quantity of bioadhesive sealant has been delivered.

13. The syringe of claim 12, wherein the at least one tab is configured to allow the at least one plunger to move relative to the at least one barrel to deliver a second predetermined quantity of bioadhesive sealant upon a force being applied to the at least one tab.

14. A bioadhesive sealant delivery system, comprising:
a manifold comprising:
an inlet;
a first outlet;
a second outlet;
at least one valve positioned and configured to facilitate selective fluid communication between the inlet and the first outlet and the inlet and the second outlet;
a syringe coupled to the inlet;
a catheter comprising:
a first lumen coupled to the first outlet;
a second lumen coupled to the second outlet.

15. The bioadhesive sealant delivery system of claim 14, wherein the catheter is configured to deliver a first volume of bioadhesive sealant through the first lumen to a vessel puncture, and deliver a second volume of bioadhesive sealant through the second lumen to the vessel puncture.

16. The bioadhesive sealant delivery system of claim 15, wherein the catheter comprises an expandable member configured to temporarily seal the vessel puncture.

17. The bioadhesive sealant delivery system of claim 14, wherein the inlet comprises a first flow path and a second flow path, the first flow path being separate from the second flow path.

18. The bioadhesive sealant delivery system of claim 17, wherein the first outlet comprises a first flow path and a second flow path and the second outlet comprises a first flow path and a second flow path, the first flow path of each of the first and second outlets positioned for selective fluid communication with the first flow path of the inlet and the second flow path of each of the first and second outlets positioned for selective fluid communication with the second flow path of the inlet.

19. The bioadhesive sealant delivery system of claim 18, wherein the first outlet comprises a mixing chamber in fluid communication with the first and second flow paths of the first outlet, and the second outlet comprises a mixing chamber in fluid communication with the first and second flow paths of the second outlet.

20. A method of delivering bioadhesive sealant, the method comprising:
delivering a first quantity of bioadhesive sealant from an inlet of a manifold to a first outlet of the manifold;
operating a valve of the manifold;
delivering a second quantity of bioadhesive sealant from the inlet of the manifold to a second outlet of the manifold.

21. The method of claim 20, further comprising:
delivering the first quantity of bioadhesive sealant from the first outlet of the manifold to a first lumen of a catheter;
delivering the second quantity of bioadhesive sealant from the second outlet of the manifold to a second lumen of the catheter.

22. The method of claim 21, further comprising:
delivering the first quantity of bioadhesive sealant from a syringe to the inlet of the manifold;
delivering the second quantity of bioadhesive sealant from the syringe to the inlet of the manifold.

23. The method of claim 22, further comprising depressing at least one tab of the syringe after delivering the first quantity of bioadhesive sealant and before delivering the second quantity of bioadhesive sealant.

24. The method of claim 20, wherein operating the valve of the manifold comprises sliding a first portion of the manifold relative to a second portion of the manifold.

25. The method of claim 20, wherein operating the valve of the manifold comprises rotating a first portion of the manifold relative to a second portion of the manifold.
